(19) United States
(12) Patent Application Publication
(10) Pub. No.: US 2022/0267728 A1
(43) Pub. Date: Aug. 25, 2022

(54) ALLOGENIC CAR-T CELL THERAPY

(71) Applicant: TX MEDIC AB, Viken (SE)

(72) Inventor: Adam BRUCE, Viken (SE)

(21) Appl. No.: 17/617,980

(22) PCT Filed: Jun. 17, 2020
(86) PCT No.: PCT/SE2020/050630

§ 371 Date: Dec. 10, 2021

(30) Foreign Application Priority Data
Jun. 18, 2019 (SE) 1950746-6

(51) Int. Cl. C12N 5/0783; A61K 31/721; A61K 35/17; A61K 9/00; A61K 9/08

(52) U.S. Cl. CPC C12N 5/0637; A61K 31/721; A61K 35/17; A61K 9/0019; A61K 9/08; C12N 2501/90

(57) ABSTRACT

The invention relates to the use of dextran sulfate, or a pharmaceutically acceptable salt thereof, in modulating leukocyte activation in allogenic CAR-T cell therapy. Dextran sulfate can be used together with allogenic CAR-T cells to achieve an activation pattern similar to what is obtained in autologous CAR-T cells therapy. Hence, dextran sulfate, or the pharmaceutically acceptable salt thereof, is capable of suppressing unspecific leukocyte activation in connection with allogenic CAR-T cell therapy.

### TECHNICAL FIELD

The present invention generally relates to allogenic CAR-T cell therapy, and in particular to the modulation of leukocyte activation in connection with allogenic CAR-T cell therapy.

### BACKGROUND

Chimeric antigen receptor (CAR) T cells are T cells that have been genetically engineered to produce an artificial T-cell receptor. CARs, also known as chimeric immunoreceptors, chimeric T cell receptors or artificial T cell receptors, are receptor proteins that have been engineered to give T cells the ability to target a specific antigen. The receptors are chimeric because they combine both antigen-binding and T-cell activating functions into a single receptor. In more detail, CARs are generally composed of three regions or domains: an ectodomain, a transmembrane domain, and an endodomain.

The ectodomain is the region of the receptor that is exposed to the outside of the T cell and interacts with potential target molecules, i.e., antigens. It generally consists of three major components, an antigen recognition region that binds the antigen, a signal peptide that directs the receptor protein into the endoplasmic reticulum, and a spacer that makes the receptor more available for binding. The antigen recognition region is responsible for targeting the CAR-T cell to cancer or tumor cells expressing a particular antigen, and typically consists of a single-chain variable fragment (scFv). An scFv is a chimeric protein made up of the light (VL) and heavy (VH) chains of immunoglobins, connected with a short linker peptide. These VL and VH regions are selected in advance for their binding ability to the target antigen. The linker between the two chains consists of hydrophilic residues with stretches of glycine and serine in it for flexibility as well as stretches of glutamate and lysine for added solubility. The spacer is a small structural domain that sits between the antigen recognition region and the outer membrane of the T cell. An ideal spacer enhances the flexibility of the scFv receptor head, reducing the spatial constraints between the CAR and its target antigen. This promotes antigen binding and synapse formation between the CAR-T cells and cancer cells. Spacers are often based on hinge domains from immunoglobulin G (IgG) or cluster of differentiation 8 (CD8).

The transmembrane domain is a structural component consisting of a hydrophobic alpha helix that spans the cell membrane. This domain is important for the stability of the receptor as a whole. Generally, the transmembrane domain from the most membrane-proximal component of the endodomain is used. The CD28 transmembrane domain is known to result in a highly expressed, stable receptor.

After an antigen is bound to the external antigen recognition region, CAR receptors cluster together and transmit an activation signal. The endodomain is the internal cytoplasmic end of the receptor that perpetuates signaling inside the T cell. Normal T cell activation relies on the phosphorylation of immunoreceptor tyrosine-based activation motifs (ITAMs) present in the cytoplasmic domain of CD3ζ. To mimic this process, the cytoplasmic domain of CD3ζ is commonly used as the primary CAR endodomain component.

T cells also require co-stimulatory molecules in addition to CD3 signaling in order to activate. For this reason, the endodomains of CAR receptors typically also include one or more chimeric domains from co-stimulatory proteins, such as CD28, 4-1BB (also known as CD137), or OX40.

CAR-T cell therapy has used various antigens, depending on which particular cancer type to treat. Examples of such antigens include CD19 used in B-cell derived cancers, such as acute lymphoblastic leukemia (ALL) and diffuse large B-cell lymphoma (DLBCL); CD30 used in refractory Hodgkin's lymphoma; CD33, CD123, and fms like tyrosine kinase 3 (FLT3) (also known as CD135) used in acute myeloid leukemia (AML); and B-cell maturation antigen (BCMA) used in multiple myeloma.

CAR-T cells can be derived either from T cells obtained from the patient's own blood, i.e., so-called autologous CAR-T cells, or derived from T cells of a donor, i.e., so-called allogeneic CAR-T cells. Autologous T cells have been the main focus in the early development of CAR-T cell therapy. However, autologous CAR-T cell therapy is marred by several shortcomings. Firstly, the cost of manufacturing a product made for an individual patient is very high. For instance, the first FDA approved patient-derived, i.e., autologous, CAR-T cell product was priced at 475,000 USD per patient. Secondly, it is not always possible to harvest sufficient number of T cells from the patient, in particular for cancer patients that may be lymphopenic from their disease or previous chemotherapy. Further potential problems include product variability and quality control, disease progression during manufacture of the autologous CAR-T cells, risk of contamination with tumor cells and T cell dysfunction.

As a consequence of these shortcomings with autologous CAR-T cell therapy, allogenic CAR-T cell therapy has achieved more focus lately. Concerns with allogenic CAR-T cell therapy have been graft versus host disease (GVHD) and rejection of CAR-T cells due to human leukocyte antigen (HLA) mismatch between the donor and the patient and unspecific leukocyte activation. Allogenic CAR-T cell therapy has the potential to be used in more cancer patients as compared to autologous CAR-T cells. There is, however, a need to improve allogenic CAR-T cell therapy in particular with regard to suppressing unspecific leukocyte activation in order to make the allogenic CAR-T cell therapy safer and more accessible.

### SUMMARY

It is a general objective to provide an improved allogenic CAR-T cells therapy.

This and other objectives are met by embodiments as disclosed herein.

The present invention is defined in the independent claims. Further embodiments of the invention are defined in the dependent claims.

An aspect of the embodiments relates to an in vitro method of modulating leukocyte activation in allogenic CAR-T cell therapy. The method comprises contacting in vitro allogenic CAR-T cells with dextran sulfate, or a pharmaceutically acceptable salt thereof, to induce a modulation in leukocyte activation in a subject administered the CAR-T cells.

Another aspect of the embodiments relates to dextran sulfate, or a pharmaceutically acceptable salt thereof, for use in inhibiting unspecific leukocyte activation causing damages to a subject treated with allogenic CAR-T cells.

Yet other aspects of the embodiments relates to dextran sulfate, or a pharmaceutically acceptable salt thereof, for use in combination with allogenic CAR-T cells in treatment of cancer, in CAR-T cell therapy, in treatment of transplant rejection, in treatment of a virus or bacterial infection, in treatment of an autoimmune disease or in treatment of systemic lupus erythematosus (SLE).

Further aspects of the embodiments relate to a composition comprising dextran sulfate, or a pharmaceutically acceptable salt thereof, and allogenic CAR-T cells, such a composition for use as a medicament, for use in allogenic CAR-T cell therapy, for use in treatment of cancer, in treatment of transplant rejection, in treatment of a virus or bacterial infection, in treatment of an autoimmune disease or in treatment of SLE.

Dextran sulfate, or the pharmaceutically acceptable salt thereof, is able to modulate leukocyte activation in allogenic CAR-T cell therapy to reduce levels of unspecific leukocyte activation, such as seen in levels of monocyte and granulocyte activation, and to achieve an activation pattern in CAR-T cells, such as seen in the activation markers CD69 and CD107a, that is similar to the ones obtained with autologous CAR-T cells. Dextran sulfate achieves this modulation without any negative effects on the CAR-T cells or the functionality of the CAR-T cells in terms of being capable of destroying target cells.

### BRIEF DESCRIPTION OF THE DRAWINGS

The embodiments, together with further objects and advantages thereof, may best be understood by making reference to the following description taken together with the accompanying drawings, in which:

FIGS. 1A to 1C illustrate percentage of cells positive for CAR-T specific marker out of the whole T cell population (CD3+ T cells, FIG. 1A), CD4+ T cells (FIG. 1B) and CD8+ T cells (FIG. 1C). Peripheral blood mononuclear cells (PBMCs) from four donors (D1-D4) were isolated, cultured on cell culture plates with OKT-3 and stimulated with IL-2 before transduction with retroviruses (2G and Mock for control). After 7 to 13 days of expansion, cells were harvested and frozen. In whole blood loop assay, CAR-T cells from D2 were used. Donor D2 was called for blood donation as the autologous blood donor. An additional donor was recruited for blood collection as the allogenic blood donor.

FIGS. 2A to 2G illustrate platelet (PLT) counts (FIG. 2A), red blood cell (RBC) counts (FIG. 2B), white blood cell (WBC) counts (FIG. 2C), lymphocyte (lymph #) counts (FIG. 2D), neutrophil (neut #) counts (FIG. 2E), monocyte (mono #) counts (FIG. 2F) and eosinophil (eo #) counts (FIG. 2G). Blood was extracted from loops at zero, 10 min, 30 min and 60 min time-points and platelets and red blood cells were automatically counted using a Sysmex XN-L 350 Hematology Analyzer.

FIGS. 3A to 3I illustrate the percentage of CD69+(FIG. 3A), CD107a+(FIG. 3B) and viability dye-positive cells (dead cells) (FIG. 3C) out of all T cells (CD3+), the percentage of CD69+(FIG. 3D), CD107a+(FIG. 3E) and viability dye-positive cells (dead cells) (FIG. 3F) in the CD8+ T-cell population and the percentage of CD69+(FIG. 3G), CD107a+(FIG. 3H) and viability dye-positive cells (dead cells) (FIG. 3I) in the CD4+ T-cell population in blood samples from an autologous donor and an allogenic donor. Fresh blood was collected and immediately mixed with ethylenediaminetetraacetic acid (EDTA) (zero time point samples) or added to loops followed by sampling and mixing with EDTA at 60 min time-point.

FIGS. 4A to 4C illustrate the percentage of cells positive for the CAR-T cell specific marker in the T cell population (CD3+) (FIG. 4A), in the CD8+ T cell population (FIG. 4B) and in the CD4+ T cell population (FIG. 4C) in blood samples from an autologous donor and an allogenic donor. Fresh blood was collected and immediately mixed with EDTA (zero time point samples) or added to loops followed by sampling and mixing with EDTA at 60 min time-point.

FIGS. 5A to 5C illustrate the percentage of CD69+(FIG. 5A), CD107a+(FIG. 5B) and viability dye-negative cells (live cells) (FIG. 5C) in the CAR-T cell population in blood samples from an autologous donor and an allogenic donor. Fresh blood was collected and immediately mixed with EDTA (zero time point samples) or added to loops followed by sampling and mixing with EDTA at 60 min time-point.

FIG. 6 illustrates the percentage of CD69+ in the CAR-T cell population in blood samples from the same autologous donor as in FIG. 5A and another allogenic donor as compared to FIG. 5A. Fresh blood was collected and immediately mixed with EDTA (zero time point samples) or added to loops followed by sampling and mixing with EDTA at 60 min time-point.

FIGS. 7A and 7B illustrate the percentage of CD69+(FIG. 7A) and CD107a+(FIG. 7B) cells in the B cell population (CD19+) in blood samples from an autologous donor and an allogenic donor. Fresh blood was collected and immediately mixed with EDTA (zero time point samples) or added to loops followed by sampling and mixing with EDTA at 60 min time-point.

FIGS. 8A and 8B illustrate the percentage of cells positive for a CD20 marker in the B cell population (CD19) (FIG. 8A) and the frequency of B cells of all lymphocytes shown as fold change over the zero sample (FIG. 8B). Analysis on blood samples from an autologous donor and an allogenic donor. Fresh blood was collected and immediately mixed with EDTA (zero time point samples) or added to loops followed by sampling and mixing with EDTA at 60 min time-point.

FIGS. 9A and 9B illustrate the percentage of CD11b+ cells in the monocyte population (FIG. 9A) and in the granulocyte population (FIG. 9B). Analysis on blood samples from an autologous donor and an allogenic donor. Fresh blood was collected and immediately mixed with EDTA (zero time point samples) or added to loops followed by sampling and mixing with EDTA at 60 min time-point.

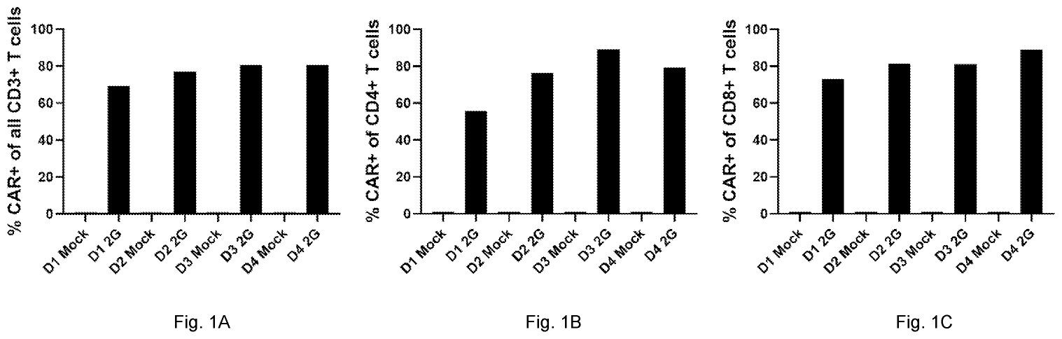

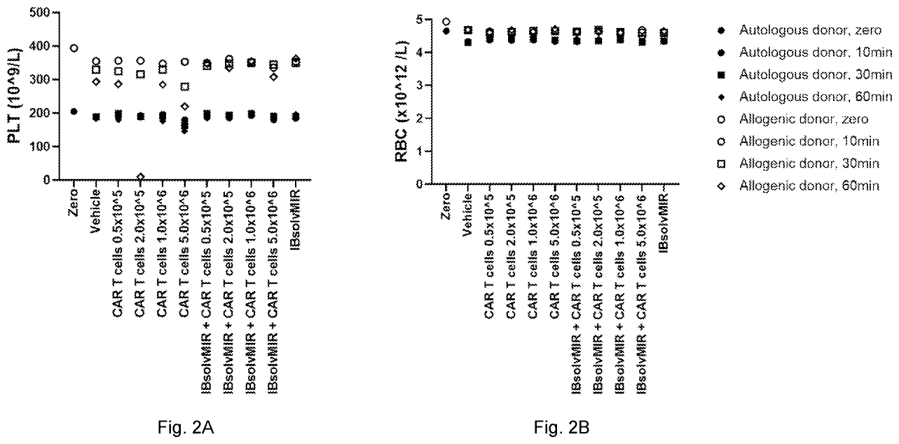

### DETAILED DESCRIPTION

The present invention generally relates to allogenic CAR-T cell therapy, and in particular to the modulation of leukocyte activation in connection with allogenic CAR-T cell therapy.

Allogenic CAR-T cell therapy is emerging as an alternative to autologous CAR-T cell therapy mainly due to the high costs in autologous CAR-T cell therapy and harvest and manufacturing failures that are common in lymphopenic patients.

A potential problem with allogenic CAR-T cell therapy is unspecific leukocyte activation, sometimes referred to as undesirable leukocyte activation, that may cause damages to both the receiving patient and to the allogenic CAR-T cells administered to the patient. The levels of such unspecific leukocyte activation are generally believed to be dependent on the degree of HLA matching between the donor and the patient, with typically more unspecific leukocyte activation in cases with poor HLA matching. The unspecific leukocyte activation may cause damages to the patient through various mechanisms including, but not limited to, cytokine release syndrome (CRS), neurologic toxicity, on target/off tumor recognition, graft versus host disease (GVHD) and anaphylaxis.

CRS is a negative immune activation resulting in elevated inflammatory cytokines. Clinical features include high fever, malaise, fatigue, myalgia, nausea, anorexia, tachycardia/hypotension, capillary leak, cardiac dysfunction, renal impairment, hepatic failure, and disseminated intravascular coagulation. The development of neurologic toxicities caused by unspecific leukocyte activation includes confusion, delirium, expressive aphasia, obtundation, myoclonus, and seizure has been reported in patients receiving CAR-T cells.

The ideal target antigen is restricted to the tumor cell and provides a critical survival signal for the malignant clone. Unfortunately, most targets of CAR T cells have shared expression on normal tissues and some degree of “on-target/off-tumor” toxicity occurs through engagement of target antigen on nonpathogenic tissues. The severity of reported events has ranged from manageable lineage depletion (B-cell aplasia) to severe toxicity (death).

There is therefore generally a need to suppress or inhibit such unspecific leukocyte activation in allogenic CAR-T cell therapy and obtain an activation pattern as seen in various activation markers, such as CD69 and CD107a, that is more similar to the activation pattern obtained with autologous CAR-T cell therapy.

Experimental data as presented herein indicate that dextran sulfate was able to modulate the leukocyte activation in allogenic CAR-T cell therapy to reduce levels of unspecific leukocyte activation, such as seen in levels of monocyte and granulocyte activation, and to achieve an activation pattern in CAR-T cells, such as seen in the activation markers CD69 and CD107a, that was similar to the ones obtained with autologous CAR-T cells.

Dextran sulfate achieved this modulation without any negative effects on the CAR-T cells or the functionality of the CAR-T cells in terms of being capable of decreasing B cell counts using a CAR with an antigen recognition region targeting the B cell antigen CD19.

An aspect of the embodiments therefore relates to an in vitro method of modulating leukocyte activation in allogenic CAR-T cell therapy. The method comprises contacting, preferably in vitro, allogenic CAR-T cells with dextran sulfate, or a pharmaceutically acceptable salt thereof, to induce a modulation in leukocyte activation in a subject administered the allogenic CAR-T cells.

Thus, dextran sulfate, or the pharmaceutically acceptable salt thereof, is contacted with the allogenic CAR-T cells and more preferably, the allogenic CAR-T cells are contacted in vitro with dextran sulfate, or the pharmaceutically acceptable salt thereof. In an embodiment, dextran sulfate, or the pharmaceutically acceptable salt thereof, could be added to a solution or vehicle comprising the allogenic CAR-T cells. In such a case, the allogenic CAR-T cells are treated with dextran sulfate, or the pharmaceutically acceptable salt thereof, prior to being administered to the patient undergoing allogenic CAR-T cell therapy.

For instance, dextran sulfate, or the pharmaceutically acceptable salt thereof, could be added to an intravenous solution bag or infusion bag comprising the allogenic CAR-T cells in an infusion solution or vehicle. The dextran sulfate, or the pharmaceutically acceptable salt thereof, may be added to such a bag in connection with or substantially prior to administering the allogenic CAR-T cells in the solution or vehicle to a subject. Alternatively, the intravenous solution bag or infusion bag could be pre-manufactured with a solution or vehicle comprising the dextran sulfate, or the pharmaceutically acceptable salt thereof, and the allogenic CAR-T cells may then be added to the bag and the solution and vehicle contained therein. A further alternative is to have a manufactured intravenous solution bag or infusion bag comprising both the dextran sulfate, or the pharmaceutically acceptable salt thereof, and the allogenic CAR-T cells.

In a generally less preferred embodiment, the allogenic CAR-T cells and the dextran sulfate, or the pharmaceutically acceptable salt thereof, could be administered separately to the patient to then contact the allogenic CAR-T cells with the dextran sulfate, or the pharmaceutically acceptable salt thereof, in vivo in the patient's body, such as in the blood system. In such a case, the allogenic CAR-T cells and the dextran sulfate, or the pharmaceutically acceptable salt thereof, are preferably administered to a same or substantially same site in the patient body or, in the case of a systemic administration, such as intravenous injection, the allogenic CAR-T cells and the dextran sulfate, or the pharmaceutically acceptable salt thereof, are preferably both administered using the same systemic route, such as both being intravenously injected.

This embodiment then relates to a method of modulating leukocyte activation in allogenic CAR-T cell therapy. The method comprises administering allogenic CAR-T cells and dextran sulfate, or a pharmaceutically acceptable salt thereof, to a subject to induce a modulation in leukocyte activation in the subject following administration of the allogenic CAR-T cells.

In an embodiment, the allogenic CAR-T cells are contacted, preferably in vitro, with the dextran sulfate, or the pharmaceutically acceptable salt thereof, to reduce activation of monocytes and/or granulocytes in the subject administered the allogenic CAR-T cells. Hence, dextran sulfate, or the pharmaceutically acceptable salt thereof, is capable of reducing or suppressing unspecific leukocyte activation in terms of being capable of reducing or suppressing activation of monocytes and/or granulocytes in the subject undergoing allogenic CAR-T cell therapy.

In an embodiment, the allogenic CAR-T cells are contacted, preferably in vitro, with the dextran sulfate, or the pharmaceutically acceptable salt thereof, to induce a leukocyte activation in the subject administered the allogenic CAR-T cells corresponding to a leukocyte activation obtained in the subject following administration of autologous CAR-T cells. Thus, the dextran sulfate, or the pharmaceutically acceptable salt thereof, is capable of achieving an activation pattern as assessed using various activation markers, preferably CD69 and/or CD107a, obtained in autologous CAR-T cell therapy even though the subject is administered allogenic CAR-T cells. The dextran sulfate, or the pharmaceutically acceptable salt thereof, could thereby been seen as “normalizing” the leukocyte activation and the activation pattern to levels generally obtained in autologous CAR-T cell therapy. Hence, in a particular embodiment, the allogenic CAR-T cells are contacted, preferably in vitro, with the dextran sulfate, or the pharmaceutically acceptable salt thereof, to induce a CAR-T cell activation in the subject administered the allogenic CAR-T cells corresponding to a CAR-T cell activation obtained in the subject following administration of autologous CAR-T cells. In an embodiment, the CAR-T cell activation is represented by level of at least one activation marker selected from the group consisting of CD69 and CD107a.

The allogenic CAR-T cells could be obtained using various known CAR-T cell manufacturing processes. For instance, the allogenic CAR-T cells can be manufactured from allogenic hematopoietic stem cell transplant (HSCT) donors. HSCT is the standard care for high risk B-ALL patients with an HLA matched donor. In such a case, CAR-T cells could be derived from such an HLA-matched donor. CAR-T cells generated from such a donor are less likely to cause GVHD due to HLA-matching and, as they are identical to the previously transplanted hematopietic stem cells, they should not attack the graft. Another source of allogenic CAR-T cells is third party viral specific (VS) T cell donors. Such donors are typically only partially HLA matched, such as 1-4 alleles to the patient. Further sources include allogenic CAR-T cells derived from healthy donors and inducible pluripotent stem (iPS) derived CAR-T cells. More information of sources for allogenic CAR-T cells can be found in Graham et al., Allogenic CAR-T Cells: More than Ease of Access?, Cells 2018, 7(10): E155, the teaching of which relating to allogenic CAR-T cell sources in paragraphs 4.1 to 4.7 is hereby incorporated by reference.

The T cells used in the allogenic CAR-T cell therapy together with the dextran sulfate, or the pharmaceutically acceptable salt thereof, could be of various types including, but not limited to, cytotoxic T cells (CD8+ T cells), T helper cells (CD4+ T cells), regulatory T cells (Tregs), and any mixture or combination thereof.

The CAR receptors expressed in the CAR-T cells could be any known CAR receptor having selected antigen recognition region and suitable transmembrane domain and endodomain. Non-limiting, but illustrative, examples of antigen recognition regions include such regions, such as scFv, capable of recognizing and specifically binding to a suitable tumor associated antigen (TAA). Examples of such TAAs include CD19, CD20, CD30, CD33, CD123, FLT3 (CD135), BCMA, mucin 1 (MUC1), mesothelin (MSLN), NY-ESO-1, alpha-fetoprotein (AFP), carcinoembryonic antigen (CEA), human epidermal growth factor receptor 2 (HER2), tumor protein p53 (p53), Ras protein (RAS), melanoma-associated antigen (MAGE). Spacers in the ectodomain of the CAR receptor could, for instance, be based on the hinge domains of IgG or CD8. An illustrative example of the transmembrane domain that could be used in the CAR receptor is the CD28 transmembrane domain. The endodomain may comprise the cytoplasmic domain of CD3ζ and one or more chimeric domains from co-stimulatory proteins, such as CD28, 4-1BB (CD137), or OX40.

Another aspect of the embodiments relates to dextran sulfate, or a pharmaceutically acceptable salt thereof, for use in inhibiting unspecific leukocyte activation causing damages to a subject treated with allogenic CAR-T cells.

In an embodiment, the unspecific leukocyte activation may also, or alternatively, cause damages to the allogenic CAR-T cells, thereby reducing the effectiveness of the allogenic CAR-T cell therapy.

In an embodiment, the dextran sulfate, or the pharmaceutically acceptable salt thereof, is for use in inhibiting monocyte and/or granulocyte activation in the subject treated with the allogenic CAR-T cells.

A further aspect of the embodiments relates to dextran sulfate, or a pharmaceutically acceptable salt thereof, for use in combination with allogenic CAR-T cells in treatment of cancer.

The cancer can be any cancer type, for which CAR-T cells therapy has been proposed. In an embodiment, the cancer is selected from the from the group consisting of leukemia, preferably chronic lymphocytic leukemia (CLL), such as advanced B-cell CLL, acute lymphoblastic leukemia (ALL), such as B-cell ALL, or acute myeloid leukemia (AML); lymphoma, preferably B-cell lymphoma, such as diffuse large B-cell lymphoma (DLBCL), or Hodgkin's lymphoma; and myeloma, preferably multiple myeloma.

Allogenic CAR-T cell therapy may also find other uses than in the treatment of cancer. For instance, CAR-T cell therapy has been applied to treat, inhibit or prevent influenza A virus by using an antigen recognition region that targets an antigen from the M2 protein, and in particular the M2 ectodomain 25 (M2e), which is highly conserved across influenza A virus (Talbot et al., An Influenza Virus M2 Protein Specific Chimeric Antigen Receptor Modulates Influenza A/WSN/33 H1N1 Infection In Vivo, The Open Virology Journal 2013, 7: 28-36). Hence, allogenic CAR-T cell therapy can be used to treat virus or bacterial infections by using CAR receptors with antigen recognition regions targeting virus associated antigens or bacterial associated antigens.

CAR-T cell therapy has also been used in the treatment of various autoimmune diseases including systemic lupus erythematosus (SLE), also known simply as lupus. In such a lupus treatment, CD19-targeted CAR-T cells targeting B cells were suggested as a stable and effective strategy to treat lupus (Kansal et al., Sustained B cell depletion by CD19-targeted CAR T cells is highly effective treatment for murine lupus, Science Translational Medicine 2019, 11(482): eaav1648).

CAR-T cell therapy further finds uses in organ transplantation by preventing or at least inhibiting transplant rejection. For instance, CAR technology has been used to redirect human Tregs toward donor-MHC class I molecules, which are ubiquitously expressed in allografts. In more detail, HAL-A2-specific CARs expressed in such Tregs alleviated the autoimmune-mediated skin injury occurring in a human skin xenograft transplant model (Boardman et al., Expression of a Chimeric Antigen Receptor Specific for Donor HLA Class I Enhances the Potency of Human Regulatory T Cells in Preventing Human Skin Transplant Rejection, American Journal of Transplantation 2017, 17: 931-943).

Additional aspects of the embodiments therefore relate to dextran sulfate, or a pharmaceutically acceptable salt thereof, for use in combination with allogenic CAR-T cells in treatment of transplant rejection, virus or bacterial infections, autoimmune diseases, or SLE. In fact, the present embodiments can be applied to any known treatment using CAR-T cells by complementing the treatment with dextran sulfate, or the pharmaceutically acceptable salt thereof. Hence, a further aspect of the embodiments relates to dextran sulfate, or a pharmaceutically acceptable salt thereof, for use in combination with allogenic CAR-T cells in CAR-T cell therapy.

Yet another aspect of the embodiments relates to a composition comprising dextran sulfate, or a pharmaceutically acceptable salt thereof, and allogenic CAR-T cells.

In an embodiment, the composition also comprises an aqueous injection solution comprising the dextran sulfate, or the pharmaceutically acceptable salt thereof, and the allogenic CAR-T cells. The aqueous injection solution could be any solution that can be administered to, preferably injected into, a subject and that is compatible with the CAR-T cells and non-toxic to the subject. The aqueous injection solution could be saline, i.e., NaCl (aq), such as 0.9% NaCl saline. Another example of an aqueous injection solution is a buffer solution. Non-limiting, but illustrative, examples of such buffer solutions is a citric acid buffer, such as citric acid monohydrate (CAM) buffer, and a phosphate buffer.

The composition may be provided in an intravenous solution bag or infusion bag as discussed in the foregoing.

Related aspects of the embodiments define the composition for use as a medicament, for use in CAR-T cell therapy, for use in treatment of cancer, for use in treatment of transplant rejection, for use in treatment of virus or bacterial infections, for use in treatment of autoimmune diseases, and/or for use in treatment of SLE.

Further aspects of the invention relates to a method of treating, preventing or inhibiting, such as delaying the onset of, cancer, transplant rejection, a virus or bacterial infection, an autoimmune disease and/or SLE. The method comprising administering dextran sulfate, or a pharmaceutically acceptable salt thereof and allogenic CAR-T cells or a composition according to the invention to a subject in need thereof.

In the following, reference to (average) molecular weight and sulfur content of dextran sulfate applies also to any pharmaceutically acceptable salt of dextran sulfate. Hence, the pharmaceutically acceptable salt of dextran sulfate preferably has the average molecular weight and sulfur content as discussed in the following embodiments.

Dextran sulfate outside of the preferred ranges of the embodiments are believed to have inferior effect and/or causing negative side effects to the cells or subject.

For instance, dextran sulfate of a molecular weight exceeding 10,000 Da (10 kDa) generally has a lower effect vs. side effect profile as compared to dextran sulfate having a lower average molecular weight. This means that the maximum dose of dextran sulfate that can be safely administered to a subject is lower for larger dextran sulfate molecules (>10,000 Da) as compared to dextran sulfate molecules having an average molecular weight within the preferred ranges. As a consequence, such larger dextran sulfate molecules are less appropriate in clinical uses when the dextran sulfate is to be administered to subjects in vivo.

Dextran sulfate is a sulfated polysaccharide and in particular a sulfated glucan, i.e., polysaccharide made of many glucose molecules. Average molecular weight as defined herein indicates that individual sulfated polysaccharides may have a molecular weight different from this average molecular weight but that the average molecular weight represents the mean molecular weight of the sulfated polysaccharides. This further implies that there will be a natural distribution of molecular weights around this average molecular weight for a dextran sulfate sample.

Average molecular weight, or more correctly weight average molecular weight (M_{w}), of dextran sulfate is typically determined using indirect methods such as gel exclusion/penetration chromatography, light scattering or viscosity. Determination of average molecular weight using such indirect methods will depend on a number of factors, including choice of column and eluent, flow rate, calibration procedures, etc.

Weight average molecular weight (M_{w}):

$$\frac{\sum {M}_{i}^{2} {N}_{i}}{\sum {M}_{i} {N}_{i}} ,$$

typical for methods sensitive to molecular size rather than numerical value, e.g., light scattering and size exclusion chromatography (SEC) methods. If a normal distribution is assumed, then a same weight on each side of M_{w}, i.e., the total weight of dextran sulfate molecules in the sample having a molecular weight below M_{w} is equal to the total weight of dextran sulfate molecules in the sample having a molecular weight above M_{w}. The parameter Nᵢ indicates the number of dextran sulfate molecules having a molecular weight of Mᵢ in a sample or batch.

In an embodiment, the dextran sulfate or the pharmaceutically acceptable salt thereof has a M_{w} equal to or below 10,000 Da. In a particular embodiment, the dextran sulfate or the pharmaceutically acceptable salt thereof has a M_{w} within an interval of from 2,000 Da to 10,000 Da.

In another embodiment, the dextran sulfate or the pharmaceutically acceptable salt thereof has a M_{w} within an interval of from 2,500 Da to 10,000 Da, preferably within an interval of from 3,000 Da to 10,000 Da. In a particular embodiment, the dextran sulfate or the pharmaceutically acceptable salt thereof has a M_{w} within an interval of from 3,500 Da to 9,500 Da, such as within an interval of from 3,500 Da to 8,000 Da.

In another particular embodiment, the dextran sulfate or the pharmaceutically acceptable salt thereof has a M_{w} within an interval of from 4,500 Da to 7,500 Da, such as within an interval of from 4,500 Da and 5,500 Da.

Thus, in some embodiments, the dextran sulfate or the pharmaceutically acceptable salt thereof has a M_{w} equal to or below 10,000 Da, equal to or below 9,500 Da, equal to or below 9,000 Da, equal to or below 8,500 Da, equal to or below 8,000 Da, equal to or below 7,500 Da, equal to or below 7,000 Da, equal to or below 6,500 Da, equal to or below 6,000 Da, or equal to or below 5,500 Da.

In some embodiments, the dextran sulfate or the pharmaceutically acceptable salt thereof has a M_{w} equal to or above 1,000 Da, equal to or above 1,500 Da, equal to or above 2,000 Da, equal to or above 2,500 Da, equal to or above 3,000 Da, equal to or above 3,500 Da, equal to or above 4,000 Da. or equal to or above 4,500 Da. Any of these embodiments may be combined with any of the above presented embodiments defining upper limits of the M_{w}, such combined with the upper limit of equal to or below 10,000 Da.

In a particular embodiment, the M_{w} of dextran sulfate, or the pharmaceutically acceptable salt thereof, as presented above is average M_{w}, and preferably determined by gel exclusion/penetration chromatography, size exclusion chromatography, light scattering or viscosity-based methods. Number average molecular weight (Mₙ):

$$\frac{\sum {M}_{i} {N}_{i}}{\sum {N}_{i}} ,$$

typically derived by end group assays, e.g., nuclear magnetic resonance (NMR) spectroscopy or chromatography. If a normal distribution is assumed, then a same number of dextran sulfate molecules can be found on each side of Mₙ, i.e., the number of dextran sulfate molecules in the sample having a molecular weight below Mₙ is equal to the number of dextran sulfate molecules in the sample having a molecular weight above Mₙ.

In an embodiment, the dextran sulfate, of the pharmaceutically acceptable salt thereof, has a Mₙ as measured by NMR spectroscopy within an interval of from 1,850 to 3,500 Da.

In a particular embodiment, the dextran sulfate, of the pharmaceutically acceptable salt thereof, has a Mₙ as measured by NMR spectroscopy within an interval of from 1,850 Da to 2,500 Da, preferably within an interval of from 1,850 Da to 2,300 Da, such as within an interval of from 1,850 Da to 2,000 Da.

Thus, in some embodiments, the dextran sulfate or the pharmaceutically acceptable salt thereof has a Mₙ equal to or below 3,500 Da, equal to or below 3,250 Da, equal to or below 3,000 Da, equal to or below 2,750 Da, equal to or below 2,500 Da, equal to or below 2,250 Da, or equal to or below 2,000 Da. In addition, the dextran sulfate or the pharmaceutically acceptable salt thereof has a Mₙ equal to or above 1,850 Da.

In an embodiment, the dextran sulfate, or the pharmaceutically acceptable salt thereof, has an average sulfate number per glucose unit within an interval of from 2.5 to 3.0.

In a particular embodiment, the dextran sulfate, or the pharmaceutically acceptable salt thereof, has an average sulfate number per glucose unit within an interval of from 2.5 to 2.8, preferably within an interval of from 2.6 to 2.7.

In an embodiment, the dextran sulfate, or the pharmaceutically acceptable salt thereof, has an average number of glucose units within an interval of from 4.0 to 6.0.

In a particular embodiment, the dextran sulfate, or the pharmaceutically acceptable salt thereof, has an average number of glucose units within an interval of from 4.5 to 5.5, preferably within an interval of from 5.0 to 5.2.

In an embodiment, the dextran sulfate, or the pharmaceutically acceptable salt thereof, has a Mₙ as measured by NMR spectroscopy within an interval of from 1,850 to 3,500 Da, an average sulfate number per glucose unit within an interval of from 2.5 to 3.0, and an average sulfation of C2 position in the glucose units of the dextran sulfate is at least 90%.

In an embodiment, the dextran sulfate has an average number of glucose units of about 5.1, an average sulfate number per glucose unit within an interval of from 2.6 to 2.7 and a Mₙ within an interval of from 1,850 Da and 2,000 Da.

In an embodiment, the pharmaceutically acceptable salt of dextran sulfate is a sodium salt of dextran sulfate. In a particular embodiment, the sodium salt of dextran sulfate has an average number of glucose units of about 5.1, an average sulfate number per glucose unit within an interval of from 2.6 to 2.7 and a Mₙ including the Na⁺ counter ion within an interval of from 2,100 Da to 2,300 Da.

In an embodiment, the dextran sulfate has an average number of glucose units of 5.1, an average sulfate number per glucose unit of 2.7, an average Mₙ without Na⁺ as measured by NMR spectroscopy of about 1,900-1,950 Da and an average Mₙ with Na⁺ as measured by NMR spectroscopy of about 2,200-2,250 Da.

The dextran sulfate according to the embodiments can be provided as a pharmaceutically acceptable salt of dextran sulfate, such as a sodium or potassium salt.

A currently preferred dextran sulfate is disclosed in WO 2016/076780.

The subject is preferably a mammalian subject, more preferably a primate and in particular a human subject. The dextran sulfate, or the pharmaceutically acceptable salt thereof, can, however, be used also in veterinary allogenic CAR-T cell therapies. Non-limiting example of animal subjects include primate, cat, dog, pig, horse, mouse, rat.

The dextran sulfate, or the pharmaceutically acceptable salt thereof, is preferably administered by injection to the subject and in particular by intravenous (i.v.) injection, subcutaneous (s.c.) injection or (i.p.) intraperitoneal injection, preferably i.v. or s.c. injection. Other parenteral administration routes that can be used include intramuscular and intraarticular injection. Injection of the dextran sulfate, or the pharmaceutically acceptable derivative thereof, could alternatively, or in addition, take place directly in, for instance, a tissue or organ or other site in the subject body, such as a solid tumor, at which the target effects are to take place.

The dextran sulfate, or the pharmaceutically acceptable salt thereof, of the embodiments is preferably formulated as an aqueous injection solution with a selected solvent or excipient. The solvent is advantageously an aqueous solvent and in particular a buffer solution. A non-limiting example of such a buffer solution is a citric acid buffer, such as CAM buffer, or a phosphate buffer. For instance, dextran sulfate of the embodiments can be dissolved in saline, such as 0.9% NaCl saline, and then optionally buffered with 75 mM CAM and adjusting the pH to about 5.9 using sodium hydroxide. Also non-buffered solutions are possible, including aqueous injection solutions, such as saline, i.e., NaCl (aq). Furthermore, other buffer systems than CAM could be used if a buffered solution are desired.

The embodiments are not limited to injections and other administration routes can alternatively be used including orally, nasally, bucally, rectally, dermally, tracheally, bronchially, or topically. The active compound, dextran sulfate, is then formulated with a suitable excipient or carrier that is selected based on the particular administration route.

The composition of the embodiments can be administered using any of the above described administration routes. Currently preferred administration routes include intravenous injection, in particular with leukemia, lymphoma and myeloma and other blood cancers or hematologic cancers, or local administration at the tumor site, in particular with solid tumors.

Suitable dose ranges for the dextran sulfate, or the pharmaceutically acceptable salt thereof, may vary according to the application, such as in vitro versus in vivo, the size and weight of the subject, the cancer type, and other considerations. In particular for human subjects, a possible dosage range could be from 1 μg/kg to 100 mg/kg of body weight, preferably from 10 μg/kg to 50 mg/kg of body weight.

In preferred embodiments, the dextran sulfate, or the pharmaceutically acceptable salt thereof, is formulated to be administered at a dosage in a range from 0.05 to 50 mg/kg of body weight of the subject, preferably from 0.05 or 0.1 to 40 mg/kg of body weight of the subject, and more preferably from 0.05 or 0.1 to 30 mg/kg, or 0.1 to 25 mg/kg or from 0.1 to 15 mg/kg or 0.1 to 10 mg/kg body weight of the subject.

The dextran sulfate, or the pharmaceutically acceptable derivative thereof, can be administered at a single administration occasion, such as in the form of a single bolus injection. This bolus dose can be injected quite quickly to the subject but is advantageously infused over time so that the dextran sulfate solution is infused over a few minutes of time to the patient, such as during 5 to 10 minutes.

Alternatively, the dextran sulfate, or the pharmaceutically acceptable salt thereof, can be administered at multiple, i.e., at least two, occasions during a treatment period.

The dextran sulfate, or the pharmaceutically acceptable salt thereof, can be administered together with other active agents, either sequentially, simultaneously or in the form of a composition comprising the dextran sulfate, or the pharmaceutically acceptable salt thereof, and at least one other active agent. The at least one active agent can be selected among any agent useful in any of the above-mentioned diseases, disorders or conditions.

### EXAMPLES

The objective of this Example was to investigate dextran sulfate in a human whole blood loop assay in combination with CAR-T cells. Cellular activation and viability and blood status was assessed after incubation in the human whole loop system with dextran sulfate with and without CAR-T cells.

Materials and Methods

Production of CAR-T Cells

Peripheral blood mononuclear cells (PBMCs) were isolated from healthy donors using Lymphoprep (Progen), stored in −70° C. in freezing medium (10% dimethyl sulfoxide (DMSO), 90% fetal calf serum (FCS)) and cultured in RPMI-1640 supplemented with 10% FCS and 1% penicillin/streptomycin. The PBMCs were activated with 1 μg/ml OKT-3 (Biolegend) and 200 IU/ml IL-2 (Roche) for 1 day to selectively stimulate T cells. Retronectin plates (Takara) were prepare in advance (7 μg per well, overnight at 4° C.) and incubated twice with 500 μl concentrated CD19-CAR-encoding retrovirus (2G) or Mock retrovirus, previously described in Karlsson et al., Evaluation of Intracellular Signaling Downstream Chimeric Antigen Receptors, PLOS ONE 2015, 10(12):e0144787, for 30 min at 37° C. Activated cells were transduced with 3 ml concentrated CD19-CAR-encoding retrovirus or Mock retorvirus for 2 days at 37° C. in the presence of retronectin-coated plates and 100 IU IL-2. Cells were cultured with 100 IU/ml IL-2 and expanded for 2 weeks before analysis. For analysis of CD19-CAR expression, cells were stained with 0.5 μl of anti-CAR-Dylight649 (Jackson ImmunoResearch), washed with phosphate-buffered saline (PBS) and followed by surface labeling (CD3, CD8, CD4, TF). Flow cytometry analysis was performed using Cytoflex (Beckman Coulter). Cell count and cell viability was determined using trypan blue (T-20 Counter, Bio-Rad).

Whole Blood Loop Assay

Blood from healthy donors was taken in an open system and immediately mixed with test compounds (CD19-CAR T cells and dextran sulfate (Tikomed AB, Sweden, WO 2016/076780, referred to as IBsolvMIR in the figures). Autologous setting included CAR-T cells generated from the same donor that donated whole blood. In allogeneic setting, blood and CAR-T cells were not matched (from different donors). All materials in direct contact with whole blood were surface heparinized in accordance with the manufacturers protocol (Corline, Sweden). Whole blood (2 ml) was added to PVC-tubing, which, with a surface heparinized metal connector, formed a loop. The dextran sulfate (0.2 g/L) and CAR-T cells (0.5-5×10⁶ cells) were added according to Table 1, and the loops were set to rotate on a wheel at 37° C. Blood aliquots were sampled, andeEthylenediaminetetraacetic acid (EDTA) was added to a final concentration of 10 mM to stop reactions at a given time-point. The automated hematology analyzer XP-300 or XN-350 (Sysmex) was used to assess blood cell count at different time points, while i-STAT cartridges (Abbott) were used to measure ACT kaolin time and prothrombin/INR time measurements.

The plasma samples were kept on ice, and plasma was collected by centrifugation at 2000×g at 4° C. for 20 minutes. The plasma was stored at −70° C. until the time of analysis. Complement analysis (C3a) was performed on plasma collected at various time points after assay start with ELISA kits from RayBiotech according to the manufacturers instructions. For experiments involving flow cytometry, blood samples were mixed with EDTA (final concentration of 10 mM), followed by the Fc block (BD Biosciences) and antibody master mix containing anti-human fluorochrome-labeled antibodies for surface staining (CD3, CD4, CD8, CD20, CD56, CD16, CD66b, CD14, BioLegend), including activation markers (CD107a, CD69, CD11b, Biolegend). Antibody master mix was incubated with whole blood for 30 min at 4° C., washed with PBS and analyzed using Cytoflex (Beckman Coulter).

TABLE 1
experiment setup

| | | Final | | |
|---|---|---|---|---|
| | Reagent | concentration | | Total number |
| Loop # | (20 μl) | in blood | Cells (100 μl) | of cells per loop |
| 1 | Vehicle | — | Vehicle | — |
| 2 | Vehicle | — | CAR-T cells from D2 | 0.5 × 10⁵ |
| 3 | Vehicle | — | CAR-T cells from D2 | 2.0 × 10⁵ |
| 4 | Vehicle | — | CAR-T cells from D2 | 1.0 × 10⁵ |
| 5 | Vehicle | — | CAR-T cells from D2 | 5.0 × 10⁶ |
| 6 | Dextran | 0.2 mg/ml | CAR-T cells from D2 | 0.5 × 10⁵ |
| | sulfate | | | |
| 7 | Dextran | 0.2 mg/ml | CAR-T cells from D2 | 2.0 × 10⁵ |
| | sulfate | | | |
| 8 | Dextran | 0.2 mg/ml | CAR-T cells from D2 | 1.0 × 10⁵ |
| | sulfate | | | |
| 9 | Dextran | 0.2 mg/ml | CAR-T cells from D2 | 5.0 × 10⁶ |
| | sulfate | | | |
| 10 | Dextran | 0.2 mg/ml | Vehicle | — |
| | sulfate | | | |

Results

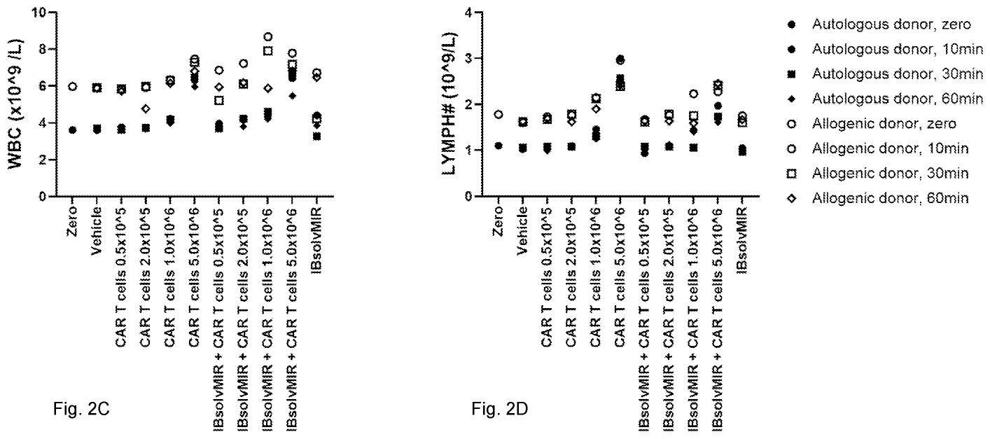

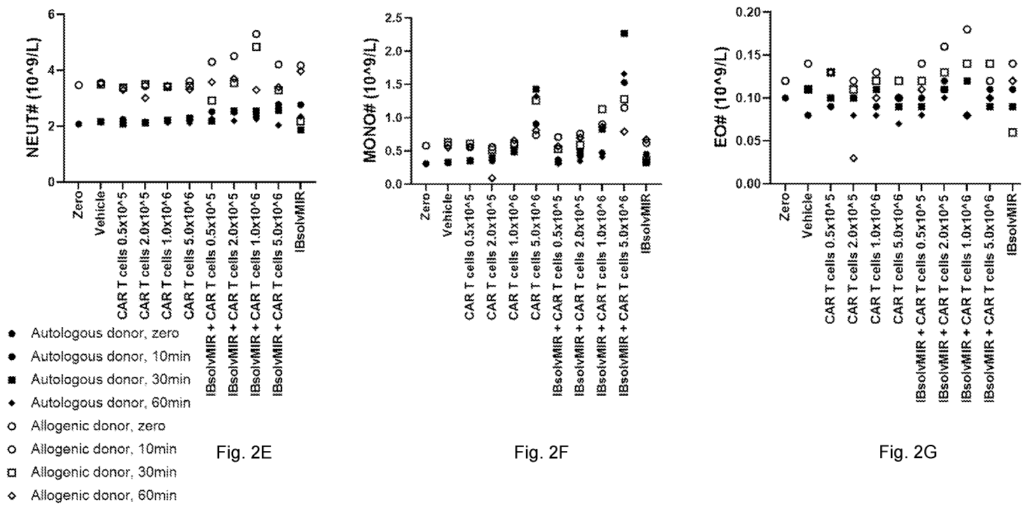

The number of platelets was within the accepted range of 20% drop compared to zero sample in vehicle samples and in the majority of samples with CAR-T cells and dextran sulfate added, indicating no platelet aggregations (FIG. 2A). The addition of CAR-T cells caused a corresponding rise of number of white blood cells (FIG. 2C) and lymphocytes (FIG. 2D) but did not affect the number of red blood cells (FIG. 2B) or eosinophils (FIG. 2G). There was a tendency of increase in neutrophils (FIG. 2E) and monocytes (FIG. 2F) at the highest CAR-T cell concentrations.

Haematocrit (HCT, %), haemoglobin (Hb, g/L), mean corpuscular volume (MVC, fL), mean corpuscular haemoglobin (MCH, pg) and mean corpuscular haemoglobin concentration (MCHC, g/L) were analyzed and found to vary <10% from zero sample for all samples (not shown).

Dextran sulfate caused a rise in all coagulation measurements (Table 2 and Table 3) and therefore had anti-coagulation properties. There was no significant difference between autologous and allogenic donor with regard to coagulation parameters.

TABLE 2
Activated clotting time (ACT), prothrombin time (PTT) and
international normalized ration (INR) for autologous donor

| | ACT | | | PTT | | | INR | | |
|---|---|---|---|---|---|---|---|---|---|
| Time point (min) | 0 | 10 | 60 | 0 | 10 | 60 | 0 | 10 | 60 |
| Zero sample | 131 | | | 12.6 | | | 1.1 | | |
| Vehicle | | 142 | ∫ | | 11.8 | 11.9 | | 1.0 | 1.0 |
| CAR-T cells | | 136 | ∫ | | 12.2 | 12.1 | | 1.0 | 1.0 |
| 0.5 × 10⁵ | | | | | | | | | |
| CAR-T cells | | 131 | ∫ | | 11.9 | 12.1 | | 1.0 | 1.0 |
| 2.0 × 10⁵ | | | | | | | | | |
| CAR-T cells | | 125 | ∫ | | 12.0 | 11.5 | | 1.0 | 1.0 |
| 1.0 × 10⁶ | | | | | | | | | |
| Dextran sulfate + | | 455 | ∫ | | 15.9 | 17.5 | | 1.3 | 1.5 |
| CAR-T cells | | | | | | | | | |
| 0.5 × 10⁵ | | | | | | | | | |
| Dextran sulfate + | | 455 | ∫ | | 17.1 | 18.4 | | 1.5 | 1.6 |
| CAR-T cells | | | | | | | | | |
| 2.0 × 10⁵ | | | | | | | | | |
| Dextran sulfate + | | 466 | ∫ | | 16.5 | 18.1 | | 1.4 | 1.5 |
| CAR-T cells | | | | | | | | | |
| 1.0 × 10⁶ | | | | | | | | | |
| Dextran sulfate | | 485 | ∫ | | 16.5 | 17.9 | | 1.4 | 1.5 |

∫ Sample coagulated before measurement

TABLE 3
Activated clotting time (ACT), prothrombin time (PTT) and
international normalized ration (INR) for allogenic donor

| | ACT | | | PTT | | | INR | | |
|---|---|---|---|---|---|---|---|---|---|
| Time point (min) | 0 | 10 | 60 | 0 | 10 | 60 | 0 | 10 | 60 |
| Zero sample | 109 | | | 13.4 | | | 1.2 | | |
| Vehicle | | * | 147 | | * | 14.3 | | * | 1.2 |
| CAR-T cells 0.5 × | | * | 142 | | * | 16.1 | | * | 1.4 |
| 10⁵ | | | | | | | | | |
| CAR-T cells 2.0 × | | * | 147 | | * | 16.1 | | * | 1.4 |
| 10⁵ | | | | | | | | | |
| CAR-T cells 1.0 × | | * | 136 | | * | 16.5 | | * | 1.4 |
| 10⁶ | | | | | | | | | |
| Dextran sulfate + | | * | 373 | | * | 28.9 | | * | 2.5 |
| CAR-T cells 0.5 × | | | | | | | | | |
| 10⁵ | | | | | | | | | |
| Dextran sulfate + | | * | 373 | | * | 33.1 | | * | 2.9 |
| CAR-T cells 2.0 × | | | | | | | | | |
| 10⁵ | | | | | | | | | |
| Dextran sulfate + | | * | 378 | | * | 31.3 | | * | 2.7 |
| CAR-T cells 1.0 × | | | | | | | | | |
| 10⁶ | | | | | | | | | |
| Dextran sulfate | | * | 356 | | * | 30.7 | | * | 2.7 |

* No coagulation detected

In general, activation of complement was seen at all time points with C3a levels slightly higher with addition of CAR-T cells in both donors (data not shown). Addition of dextran sulfate decreased levels of C3a, both alone and in co-administration with CAR-T cells (data not shown).

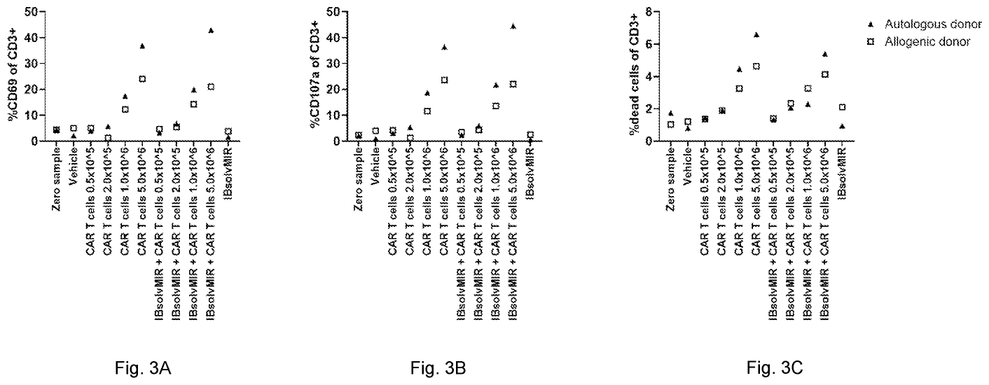

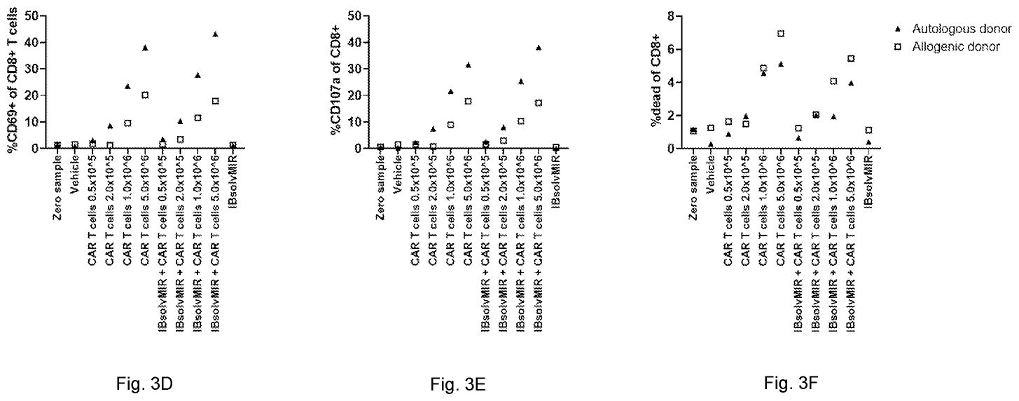

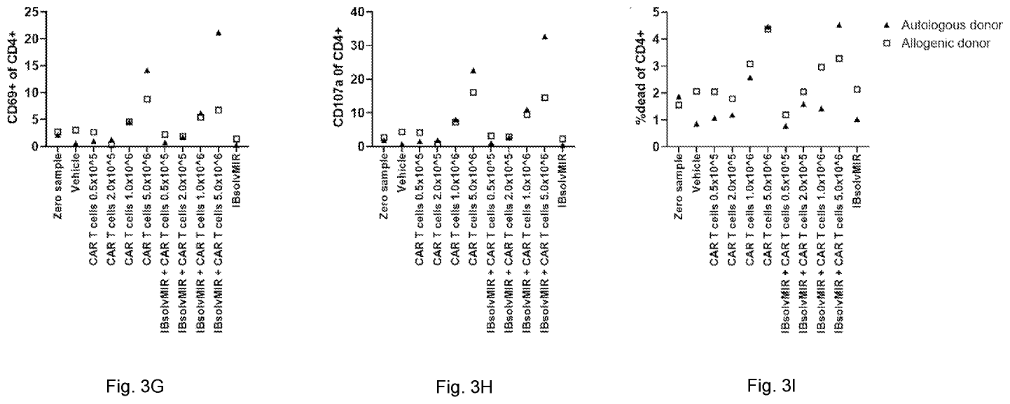

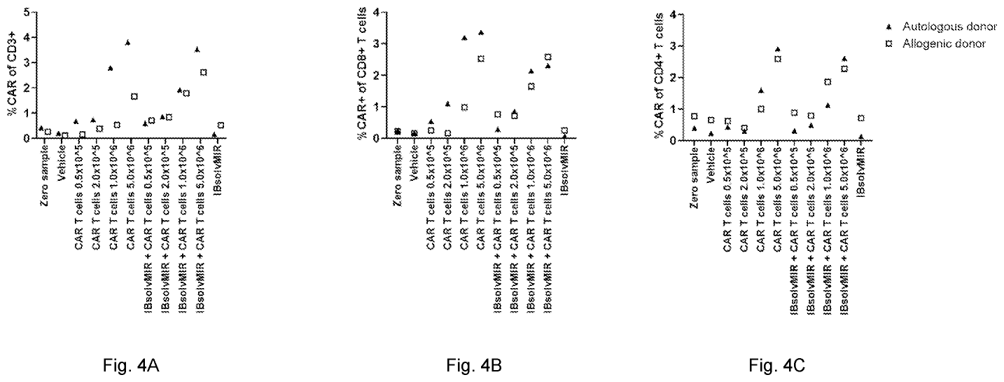

In general, viability and activation of T cell population (blood donors CD3+, CD4+, CD8+ T cells) was similar in groups with and without dextran sulfate (FIGS. 3A to 3I). Activation markers were increased in higher number of CAR-T cells infused, as expected. The proportion of CAR-T cells in T cell was higher in autologous donor than allogeneic donor (FIGS. 4A to 4C).

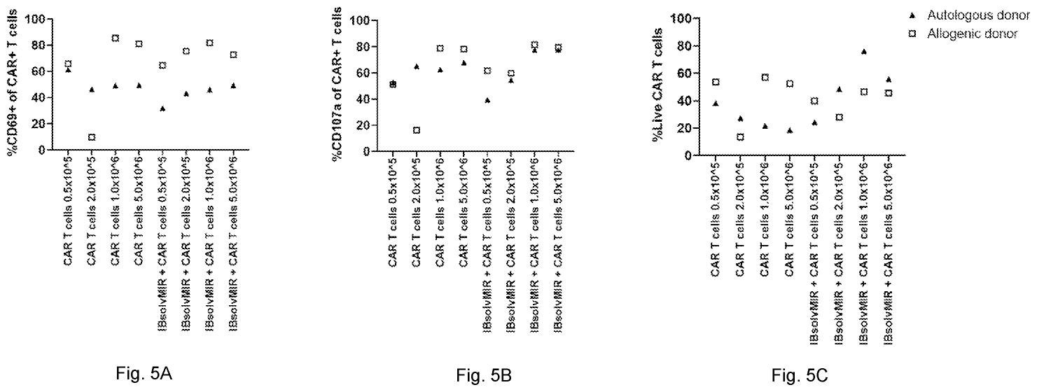

More than 90% of the CAR-T cells were viable prior addition to the whole blood loop system, measured by Bio-Rad cell counter. In autologous donor, viability of the CAR-T cells was approximately 40% without dextran sulfate and approximate 60% with dextran sulfate for the autologous donor (FIG. 5C). In the allogeneic donor, the viability of CAR-T cells was between 40-60% (FIG. 5C). Dextran sulfate increased the percentage of cells positive for the CAR-T cell specific marker in the T cell population from the allogenic donor (FIG. 4A). There was a similar trend in increase in the percentage of cells positive for the CAR-T cell specific marker in the CD8+ T cell population (FIG. 4B) and in the CD4+ T cell population (FIG. 4C) from the allogenic donor with dextran sulfate. The proportion of CD3+ CAR-T cells and CD8+ CAR-T cells were more similar between autologous and allogenic groups when dextran sulfate was added,

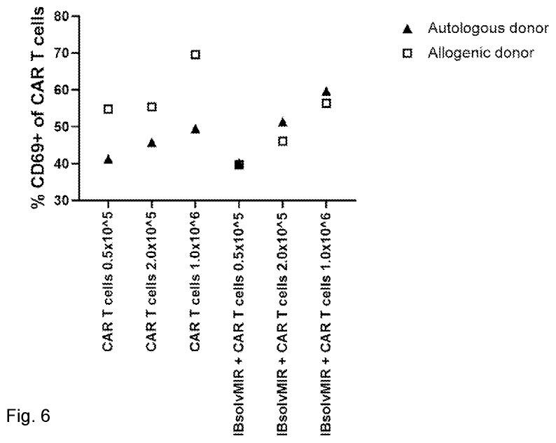

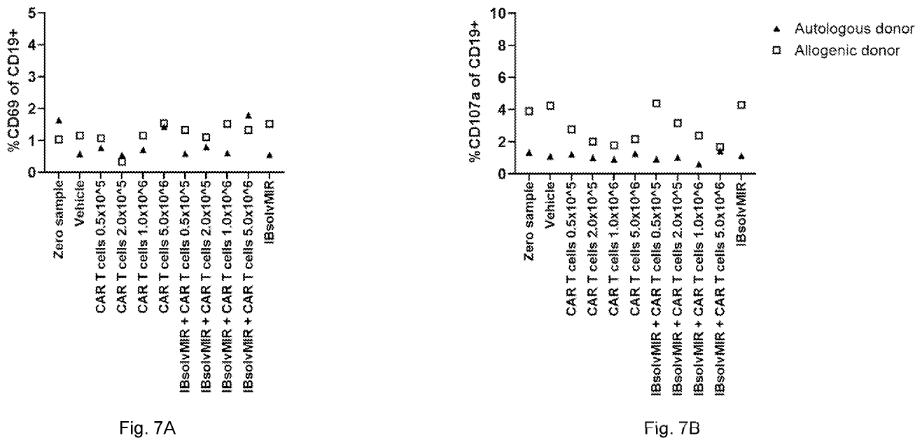

There was a significant difference in the levels of activation markers between untreated allogenic and autologous CAR-T cell groups (FIGS. 5A, 5B and 6). Dextran sulfate induced a modulation in the levels of activation markers in the allogenic CAR-T cell groups to be more similar to the levels of activation markers in the autologous CAR-T cell groups (FIGS. 5A, 5B and 6). Hence, the activation pattern between the allogenic and autologous CAR-T cell groups became more similar between the donors with dextran sulfate.

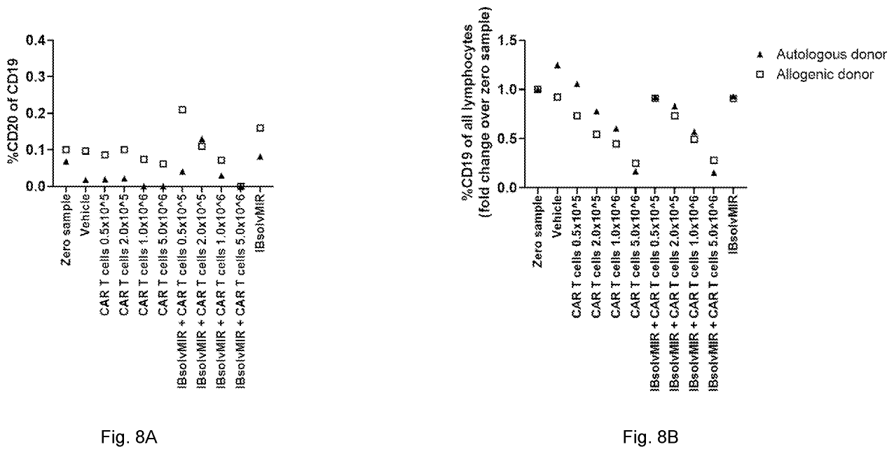

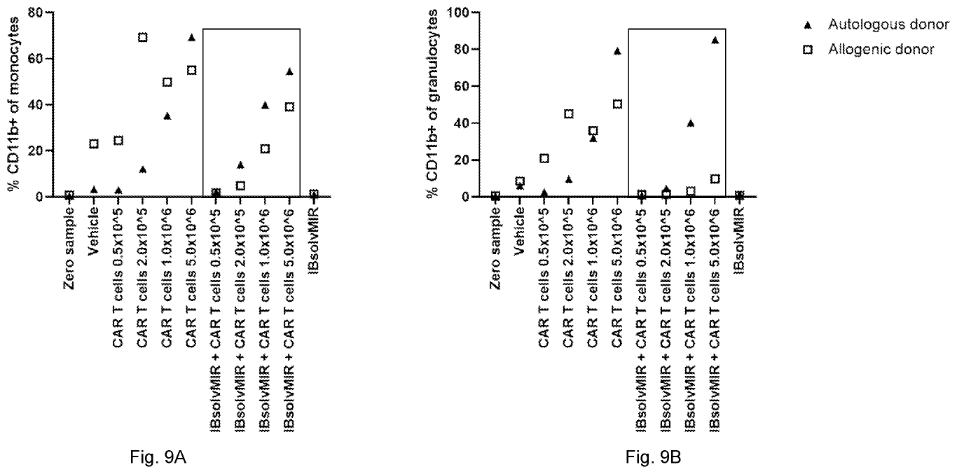

Decreased number of B cells were noted in groups with added CAR-T cells (FIGS. 8A and 8B) and there was no distinct difference in B cell decrease or activation between donors, which is expected since the decrease in B cell count is dependent on the CAR construct. Highest activation of B cells was noted in groups with highest number of CAR-T cells added (FIGS. 7A and 7B). Both B cell count and activation pattern were similar in samples with or without dextran sulfate confirming that dextran sulfate did not have any negative impact on CAR functionality.

Activation of monocytes and granulocytes was clearly increased in loops with CAR-T cells. Addition of dextran sulfate decreased expression of activation marker CD11 b on both monocytes and granulocytes (FIGS. 9A and 9B).

Dextran sulfate did not have any negative effect on the CAR-T cells in targeting B cells. Hence, the CAR functionality was not negatively affected by dextran sulfate. Dextran sulfate was capable of reducing unspecific activation of the CAR-T cells in the autologous groups to bring the activation patterns close to the activation levels as seen using autologous CAR-T cells. Furthermore, dextran sulfate was capable of reducing monocyte and granulocyte activating, which otherwise could amount to at least a portion of the unspecific leukocyte activation seen in allogenic CAR-T cell therapy.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-31. (canceled)

32. An in vitro method of modulating leukocyte activation in allogenic chimeric antigen receptor (CAR)-T cell therapy, the method comprising contacting in vitro allogenic CAR-T cells with dextran sulfate, or a pharmaceutically acceptable salt thereof, to induce a modulation in leukocyte activation in a subject administered the allogenic CAR-T cells.

33. The in vitro method according to claim 32, wherein contacting in vitro comprises contacting in vitro the allogenic CAR-T cells with the dextran sulfate, or the pharmaceutically acceptable salt thereof, to reduce activation of monocytes and/or granulocytes in the subject administered the allogenic CAR-T cells.

34. The in vitro method according to claim 32, wherein contacting in vitro comprises contacting in vitro the allogenic CAR-T cells with the dextran sulfate, or the pharmaceutically acceptable salt thereof, to induce a leukocyte activation in the subject administered the allogenic CAR-T cells corresponding to a leukocyte activation obtained in the subject following administration of autologous CAR-T cells.

35. The in vitro method according to claim 34, wherein contacting in vitro comprises contacting in vitro the allogenic CAR-T cells with the dextran sulfate, or the pharmaceutically acceptable salt thereof, to induce a CAR-T cell activation in the subject administered the allogenic CAR-T cells corresponding to a CAR-T cell activation obtained in the subject following administration of autologous CAR-T cells.

36. The in vitro method according to claim 35, wherein the CAR-T cell activation is represented by a level of at least one activation marker selected from the group consisting of CD69 and CD107a.

37. A method for inhibiting unspecific leukocyte activation, the method comprising administering dextran sulfate, or a pharmaceutically acceptable salt thereof, to a subject treated with allogenic chimeric antigen receptor (CAR)-T cells to inhibit unspecific leukocyte activation causing damages to the subject.

38. The method according to claim 37, wherein administering dextran sulfate comprises administering dextran sulfate, or the pharmaceutically acceptable salt thereof, to the subject to inhibit monocyte and/or granulocyte activation causing damages to the subject.

39. A method treating cancer, the method comprising administering dextran sulfate, or a pharmaceutically acceptable salt thereof, in combination with allogenic chimeric antigen receptor (CAR)-T cells or a composition comprising dextran sulfate, or the pharmaceutically acceptable salt thereof, and allogenic CAR-T cells to a subject suffering from cancer.

40. The method according to claim 39, wherein the cancer is selected from the group consisting of leukemia, lymphoma and myeloma.

41. The method according to claim 40, wherein
the leukemia is selected from the group consisting of chronic lymphocytic leukemia (CLL), advanced B-cell CLL, acute lymphoblastic leukemia (ALL), B-cell ALL, and acute myeloid leukemia (AML);
the lymphoma is selected from the group consisting of B-cell lymphoma, diffuse large B-cell lymphoma (DLBCL), and Hodgkin's lymphoma; and
the myeloma is multiple myeloma.

42. A method for treating transplant rejection, the method comprising administering dextran sulfate, or a pharmaceutically acceptable salt thereof, in combination with allogenic chimeric antigen receptor (CAR)-T cells or a composition comprising dextran sulfate, or the pharmaceutically acceptable salt thereof, and allogenic CAR-T cells to a subject undergoing organ transplantation.

43. A method for treating a virus or bacterial infection, the method comprising administering dextran sulfate, or a pharmaceutically acceptable salt thereof, in combination with allogenic chimeric antigen receptor (CAR)-T cells or a composition comprising dextran sulfate, or the pharmaceutically acceptable salt thereof, and allogenic CAR-T cells to a subject suffering from a virus or bacterial infection.

44. A method for treating an autoimmune disease, the method comprising administering dextran sulfate, or a pharmaceutically acceptable salt thereof, in combination with allogenic chimeric antigen receptor (CAR)-T cells a composition comprising dextran sulfate, or the pharmaceutically acceptable salt thereof, and allogenic CAR-T cells to a subject suffering from an autoimmune disease.

45. A method for treating systemic lupus erythematosus (SLE), the method comprising administering dextran sulfate, or a pharmaceutically acceptable salt thereof, in combination with allogenic chimeric antigen receptor (CAR)-T cells or a composition comprising dextran sulfate, or the pharmaceutically acceptable salt thereof, and allogenic CAR-T cells to a subject suffering from SLE.

46. A composition comprising dextran sulfate, or a pharmaceutically acceptable salt thereof, and allogenic human chimeric antigen receptor (CAR)-T cells.

47. The composition according to claim 46, further comprising an aqueous injection solution comprising the dextran sulfate, or the pharmaceutically acceptable salt thereof, and the allogenic CAR-T cells.

48. The composition according to claim 46, wherein the dextran sulfate, or the pharmaceutically acceptable salt thereof, has an average molecular weight equal to or below 10 000 Da.

49. The composition according to claim 48, wherein the dextran sulfate, or the pharmaceutically acceptable salt thereof, has an average molecular weight within a range of 2 000 and 10 000 Da.

50. The composition according to claim 49, wherein the dextran sulfate, or the pharmaceutically acceptable salt thereof, has an average molecular weight within a range of 4 500 and 7 500 Da.

51. The composition according to claim 46, wherein the dextran sulfate, or the pharmaceutically acceptable salt thereof, has an average sulfur content in a range from 15 to 20%.

52. The composition according to claim 46, wherein the dextran sulfate, or the pharmaceutically acceptable salt thereof, has a number average molecular weight (Me) as measured by nuclear magnetic resonance (NMR) spectroscopy within an interval of 1850 and 3500 Da.

53. The composition according to claim 52, wherein the dextran sulfate, or the pharmaceutically acceptable salt thereof, has a Mₙ as measured by NMR spectroscopy within an interval of 1850 and 2000 Da.

54. The composition according to claim 46, wherein the dextran sulfate, or the pharmaceutically acceptable salt thereof, has an average sulfate number per glucose unit within an interval of 2.5 and 3.0.

55. The composition according to claim 46, wherein the dextran sulfate, or the pharmaceutically acceptable salt thereof, has on average 5.1 glucose units and an average sulfate number per glucose unit of 2.6 to 2.7.

56. The composition according to claim 46, wherein the pharmaceutically acceptable salt thereof is a sodium salt of dextran sulfate.
